(19) United States
(12) Patent Application Publication
(10) Pub. No.: US 2007/0112365 A1
(43) Pub. Date: May 17, 2007

(54) Partial occlusion surgical guide clip

(75) Inventors: Nabil Hilal, Laguna Niguel, CA (US); John R. Brustad, Dana Point, CA (US); Edward D. Pingleton, San Juan Capistrano, CA (US); Ghassan Sakakine, Rancho Santa Margarita, CA (US); Charles C. Hart, Summerville, SC (US)

Correspondence Address: APPLIED MEDICAL RESOURCES CORPORATION, 22872 Avenida Empresa, Rancho Santa Margarita, CA, 92688, US

(73) Assignee: Applied Medical Resources Corporation

(21) Appl. No.: 11/280,098

(22) Filed: Nov. 15, 2005

(51) Int. Cl. A61B 17/08

(52) U.S. Cl. 606/157

(57) ABSTRACT

A surgical guide clip is provided that comprises connectors and jaws attached at each end to the connectors. The jaws being piovtable to open the jaws and having aspects of ratcheting mechanisms to provide incremental closing of the jaws together.

### Background

This invention relates generally to clips and, in particular, atraumatic occlusion guide clips and methods thereof.

Various types of surgical instruments have been developed for the occlusion and ligation of vessels as well as other conduits and tissue structures. Cross-clamping is a term often used to describe an occlusion or exclusion technique. This technique generally makes use of a large scissors-like or clamp-like device that closes compressively upon a vessel, a conduit, a body passage or an organ. These devices are generally employed to provide hemostasis while a section of tissue is surgically treated, removed or excised.

Specifically with reference to surgical procedures that involves the kidney or liver, a very large cross-clamp may be required. This is especially so, if, for instance, a large portion of tissue must be removed. If a portion of a kidney is to be removed in a partial-nephrectomey procedure, it is desirable to preserve the remaining portion of that kidney. As such, the occlusion instrument being compatible with the cutting instrument can assist in enhancing the preservation of the remaining healthy tissue or portion of an organ. Likewise, an occlusion instrument having atraumatic characteristics can assist in enhancing the preservation of the remaining tissue or portion of the organ.

Furthermore, an occlusion instrument that provides simple placement on the tissue, reliable occlusion when so placed, and simple removal as the procedure is completed can be advantageous. Moreover, since typical occlusion instruments used in open surgery are most often not suited for use in laparoscopic surgery, if the occlusion instrument allows the procedure to be performed laparoscopically, the recovery or healing time of the patient may be reduced.

### SUMMARY

The present invention, in various aspects, generally provides an occlusion atraumatic guide clip and methods thereof. In one aspect, a surgical guide clip comprises a first and second connector. The first connector has a first support and a second support with the first support connected to the second support and the second connector has a third support and a fourth support with the third support connected to the fourth support. The guide clip also has a first jaw with a tissue contacting surface, a first end and a second end. The first support extends from the first end of the first jaw. The third support extends from the second end of the first jaw and the first and third supports extend generally perpendicularly to the tissue contacting surface of the first jaw. The guide clip also has a second jaw having a tissue contacting surface, a first end and a second end. The second support extends from the first end of the second jaw. The fourth support extends from the second end of the second jaw and the second and fourth supports extend generally perpendicularly to the tissue contacting surface of the second jaw.

In another aspect, a surgical guide clip comprises a first and second jaw. The first jaw has a first end, a second end and a tissue contacting surface extending from the first end to the second end and the second jaw has a first end, a second end and a tissue contacting surface extending from the first end to the second end. The first jaw and the second jaw are movable between a spaced position and a proximate position. A first connector is coupled to the first end of the first jaw and the first end of the second jaw and has a first projection operatively engageable with a second projection to regulate movement of the first jaw and second jaw between the spaced and proximate position. A second connector is coupled to the second end of the first jaw and the second end of the second jaw. In one aspect, a first direction in which the first and second jaws are movable between the spaced and proximate position is a clamping direction or the direction traversed by the jaws closing, e.g., a generally perpendicular line or course from the tissue contacting surface of the first jaw towards the tissue contacting surface of the second jaw. In another aspect, the second direction is opposite in direction from the first direction or the reverse direction in which the jaws open or move towards the spaced position, e.g., a generally perpendicular line or course from the tissue contacting surface of the second jaw towards the tissue contacting surface of the first jaw.

In a further aspect, a surgical guide clip comprises means for atraumatically securing tissue between a first tissue contacting surface and a second contacting surface and movable between a spaced position and a proximate position, e.g., jaws, and means for controlling movement of the means for atraumatically securing tissue between the spaced position and the proximate position through a plurality of intermediate set positions between the spaced position and the proximate position, e.g., ratchet mechanisms such as a support from one jaw with a tooth engaging teeth from a support from the other jaw. The clip also comprises means for releasing the means for controlling movement to permit movement of the means for atraumatically securing tissue from the proximate position to the spaced position bypassing at least one of the plurality of intermediate set positions, e.g., pivots in the supports.

Many of the attendant features of the present invention will be more readily appreciated as the same becomes better understood by reference to the following detailed description and considered in connection with the accompanying drawings in which like reference symbols designate like parts throughout.

### BRIEF DESCRIPTION OF THE DRAWINGS

FIGS. 1A-C illustrate perspective views of an aspect of a clip in accordance with the present invention;

FIGS. 2A-B illustrate perspective views of one aspect of a clip in accordance with the present invention;

FIG. 3 illustrates a perspective view of one aspect of a clip in accordance with the present invention;

FIG. 4A illustrates a perspective view of one aspect of a clip in accordance with the present invention;

FIGS. 4B-C illustrate a perspective view of one aspect of a clip in relation to an exemplary tissue or organ in accordance with the present invention;

FIG. 5A illustrates a side view of one aspect of a clip in accordance with the present invention;

FIG. 5B illustrates a top/bottom view of one aspect of a clip in accordance with the present invention;

FIG. 5C illustrates a side view of one aspect of a clip in accordance with the present invention;

FIGS. 6A-E illustrate perspective views of one aspect of a clip in relation to an exemplary cutting device and/or tissue or organ in accordance with the present invention;

FIG. 7 illustrates a perspective view of one aspect of a clip in accordance with the present invention;

FIG. 8 illustrates a perspective view of one aspect of a clip in accordance with the present invention;

FIG. 9 illustrates a perspective view of one aspect of a clip in accordance with the present invention;

FIG. 10 illustrates a perspective view of one aspect of a clip in accordance with the present invention;

FIG. 11 illustrates a perspective view of one aspect of a clip in accordance with the present invention;

FIGS. 12A-B illustrate perspective views of one aspect of a clip in accordance with the present invention; and

FIG. 13 illustrates a side view of one aspect of a clip in accordance with the present invention.

### DETAILED DESCRIPTION

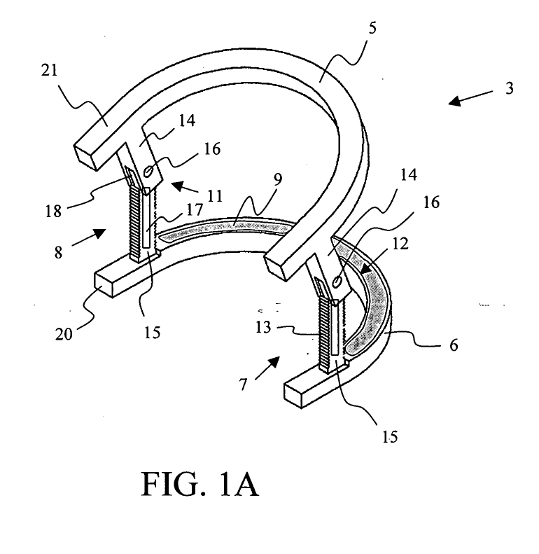

In FIG. 1A, a clip 3 is shown with a first jaw 5 and a second jaw 6. The first jaw 5 is attached at each end to the second jaw by connectors 7,8. In the aspect shown, the first and second jaws are symmetrical and have a generally curved structure and, in one aspect, have atraumatic surfaces or covers 9. The connectors 7,8 are also symmetrical and include pivot points 11, 12 which allows the first and second jaws to open and to increase the span between the jaws when opened thereby increasing the amount of tissue, organ or another object that may be clamped. The pivot points also eases the removal of the clip 3. At least one of the connectors 7, 8 in one aspect has teeth 13 that govern or provide incremental clamping or closing of the jaws together. The steps or teeth 13 and the engagement or operation of such also strengthens the connection between the jaws to ensure secure placement and occlusion.

Each connector 7, 8 has a first support 14 and a second support 15. The first support 14 from one end extends orthogonally from the first jaw 5 and on the other end is connected to the second support 15 through a pin 16. The second support 15 has a pin slot 17 or opening configured to slidably receive the pin 16. As such, the engagement of the pin 16 with the pin slot 17 connects the first and second jaws together and provides a sliding and pivoting relation between the jaws. Similar to the first support, the second support 15 extends orthogonally from the second jaw 6. The first support 14 is hollow or includes a longitudinal opening configured to receive the second support 15 as the first and second jaws are brought together. Hence, the width or diameter of the first support 14 is larger than the width or diameter of the second support 15.

In one aspect, the first support 14 includes at least one detent, step, tooth or projection extending within the inner periphery of the hollow portion or opening within the first support 14. The second support 15 includes detents, steps, teeth or projections 13 extending laterally from the second support 15 and extend longitudinally along all or a portion of the second support. When the first support 14 is substantially longitudinally aligned with the second support 15, the tooth of the first support 14 engages the teeth 13 of the second support 15. In one aspect, the second support 15 includes teeth 13 extending laterally from more than one sidewall of the second support 15 for mating or engaging with one or more teeth of the first support 14 correspondingly extending laterally from the inner periphery of the hollow portion of the first support 14. As the first and second jaws 5, 6 are closed or brought together towards a proximate position, the first and second supports 14, 15 also incrementally move together (FIGS. 1B-1C). In one aspect, each of the teeth 13 has an undercut in each mating face of the teeth that positively locks with a corresponding mating face of a tooth of the first support 14 in a set condition.

The engagement of the tooth or teeth of the first support 14 with the teeth of the second support 15 permits ratchet-like movement of the first support 14 towards the second support 15 and the second support towards the first support, but prevents backing up or reversing direction when the supports are longitudinally aligned. Therefore, this engagement provides a one-way ratchet that holds the first and second jaws in a set condition as the two jaws are brought or squeezed together by a surgeon's hand or through a surgical instrument, e.g., a grasper. As shown in FIGS. 1B-1C, the jaws can be brought together or closed to clamp the tissue in various stages, intermediate set positions, increments or amounts to partially to fully occlude the tissue there between.

The first and second jaws 5, 6 when pivoted disrupts the longitudinal alignment of the supports that causes the teeth 13 of the second support 15 to lose engagement with the tooth or teeth of the first support 14. The normal state or tendency of the clamped tissue or organ to expand causes or forces the first and second jaws 5, 6 apart or open towards a spaced position. In one aspect, the pivot motion is slight to release or open the jaws causing the jaws to respond to the pressure or decompression of the compressed tissue. A spring, in one aspect, is disposed within the hollow portion of the first support or otherwise coupled to the first or second support to bias or enhance the tendency of the first and second jaws 5, 6 to an open position.

A pivot slot or opening 18 on the first support 14 extends along a portion of a sidewall of the first support 14 to allow the first and second jaws 5, 6 to pivot in one direction, away from clamped tissue or clamping point. Once pivoted to disengage the tooth or teeth of the first support 14 from the teeth of the second support 15, the jaws may be opened. The width and length of the pivot slot 18 is sized and configured to accommodate sufficient pivoting of the jaws to release the teeth or ratchet engagement of the supports 14, 15. In one aspect, the width and length of the pivot slot 18 is approximately equal to or greater than the width and length of the second support 15 to permit the first and second jaws 5, 6 maximum separation when pivoted open. A second pivot slot (not shown), in one aspect, extends along a portion of the opposing sidewall of the first support 14 to increase the amount of pivoting of the first and second jaws 5, 6 away from each other.

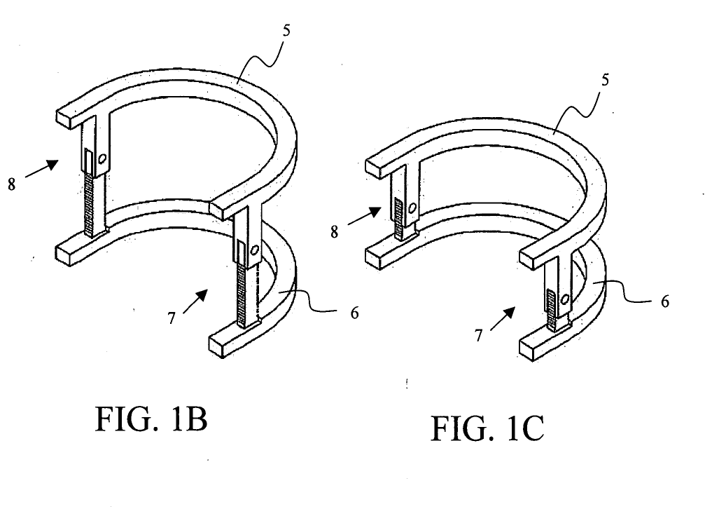

Extension actuators 20, 21 respectively extend from the first jaw 5 and second jaw 6. By squeezing the extension actuators together, the first and second jaws 5, 6 pivot releasing or opening the jaws. In one aspect, the extension actuators have grooves, apertures, slots, texture, projections or an enlarged surface area to enhance gripping or actuation of the extension actuators 20, 21. FIGS. 2A-2B provide an aspect of an occlusion clip with extension actuators 22 extending from the first jaw 5. By pulling the extension actuator 22, the first and second jaws 5, 6 pivot releasing or opening the jaws. By pushing or otherwise moving the extension actuator 22 towards the tissue to be clamped, the first and second jaws align to allow incremental closure of the jaws.

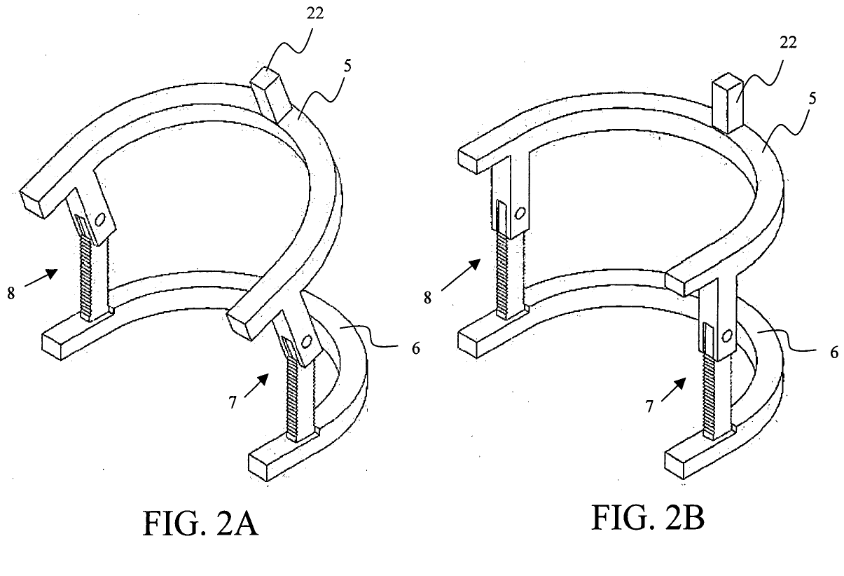

In FIG. 3 an aspect of an occlusion instrument with a reduced profile is shown. The maximum height of the clip previously described is generally defined by the maximum height of the supports and jaws including the amount of distance the jaws are permitted to open towards a spaced position. The maximum height of the clip provided in FIG. 3 is generally defined by the height of a jaw and the amount of distance the jaws are permitted to open. The clip may also be useful to reside in or enter through an access port or body cavity having minimal space or clearance. This clip and other various clips described or to be described herein may also be sized and configured to facilitate the use of the clip in a trocar or tube having a working channel with a 5-12 millimeter diameter. Also the clips provide the ability to maintain jaw symmetry and linear orientation about the axis of a grasper, cutting device or the tissue being occluded or removed which may be useful when used or manipulated through a trocar or to access a remote site.

Clip 30 comprises a first jaw 31 connected to a second jaw 32 at connectors or hinges 33, 34 on each side of the clip. In one aspect, the clip is sized and configured to be used to occlude tissue along a large lateral or horizontal plane or cross-sectional area relative to the vertical or height portion of tissue. One or both hinges 33, 34 comprises a one-way ratchet that holds the first and second jaws in a set condition through a plurality of intermediate set positions as the two jaws are brought or squeezed together towards a proximate position. The hand or fingers of a surgeon or by a surgical instrument such as a laparoscopic grasper may be used to close the jaws. The one-way ratchet as such prevents the re-opening of the jaws as induced by the compressed tissue even when the pressure to bring the jaws together is removed. The ratchet hinge or hinges, in one aspect, includes a series of the teeth or steps that radially extend from an inner spindle or stationary shaft disposed in the hinge 33, 34.

In one aspect, the hinges or connectors 33, 34 has supports extending from each corresponding jaw. The hinge 33 has a first support extending laterally or perpendicularly from one end of the first jaw 31 coupled to a second support extending laterally from one end of the second jaw 32. Likewise, the hinge 34 has a third support extending laterally or perpendicularly from another end of the first jaw 31 coupled to a fourth support extending laterally from another end of the second jaw 32. At least one tooth or step extends from the first or second jaws or from the extensions or supports extending from one or both of the jaws. The tooth is sized and configured to fit securely upon the stepped ratchet hinge or hinges. The jaws are held in a locked or set condition as the first jaw is moved to each of the steps of the hinges through one or more intermediate set positions. Each of the steps, in one aspect, has an undercut in each mating faces that positively locks with a corresponding mating face of at least one tooth in a set condition.

At least one or two release actuators or buttons 35, 36 extend from the hinge or hinges 33, 34 including a one-way ratchet. Squeezing or actuating hinge buttons 35, 36 together or in a generally inward direction releases the ratcheted hinges. This releases or disengages the ratcheted engagement and allows the pressure of the compressed tissue to force the two jaws apart or open towards a spaced position. In another aspect, at least one of the hinges comprises a two-way ratchet. The two-way ratchet holds the first and second jaws in a set condition as the two jaws are brought or squeezed together. Squeezing or actuating a release actuator together or in a generally inward direction or the two jaws apart switches or reverses the ratcheting direction and/or the ratcheted hinge engagement.

In one aspect, the hinges comprises of ratcheted wheels or pawls extending within the hinges or extending from the jaws. Also, in one aspect, the jaws comprise a leaf, lever or compression spring connected between the jaws and/or within the hinges to bias the jaws to an open position. In another aspect, frictional engagement of the hinges 33, 34 govern the closing and/or opening of the jaws.

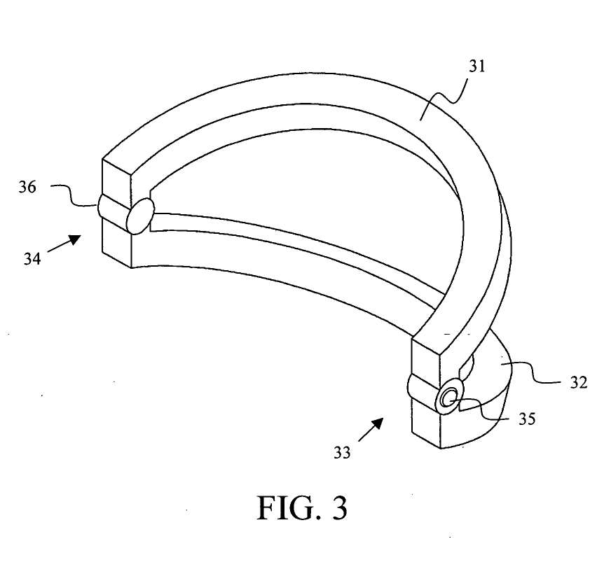

A clip 40 shown in FIGS. 4A-C comprises a first jaw 41 and a second jaw 42. FIG. 4B shows the clip with the first and second jaws 41, 42 being brought together to clamp the tissue. The first jaw 41 is attached at each end to the second jaw by connectors 43, 44. The connectors 43, 44 are slidably connected to each other longitudinally and laterally. Laterally sliding or shifting the connectors 43, 44 and thus the first and second jaws 41, 42 relative to each other allows the jaws to open towards a spaced position. Steps or teeth 47 on connectors 43, 44 govern or provide incremental clamping or closing of the jaws together towards a proximate position. As such, the connectors 43, 44 comprise a one-way ratchet that holds the first and second jaws 41, 42 through a plurality of intermediate set positions as the two jaws are brought or squeezed together.

Each connector 43, 44 has a first support 45 and a second support 46 each extending generally orthogonal to respective first jaw 41 and second jaw 42. The first support 45 is slidably engaged with the second support 46. The first support 45 is hollow or includes a longitudinal opening configured to receive the second support 46 as the first and second jaws are brought together. Hence, the width or diameter of the first support 45 is larger than the width or diameter of the second support 46. As such, the first support 45 is sized and configured to fit securely upon the stepped ratchet portions of the second support 46.

The first support 45 includes at least one detent, step, tooth or projection extending within the inner periphery of the hollow portion or opening within the first support 45. The second support 46 includes detents, steps, teeth or projections 47 extending laterally from the second support 46 and extend longitudinally along all or a portion of the second support. As the first and second jaws 5, 6 are closed or brought together towards a proximate position, the first and second supports 45, 46 also incrementally move together with the tooth of the first support 45 engaging the teeth 47 of the second support 46. In one aspect, each of the teeth 47 has an undercut in each mating face of the teeth that positively locks with a corresponding mating face of the tooth of the first support 45 in a set condition.

In one aspect, the at least one tooth is positioned along a portion of a sidewall of the first support 45 and teeth of the second support 46 is positioned along a portion of a sidewall of the second support. The tooth of the first support operatively engages with the teeth of the second support as the portion along the sidewall of the first support is aligned with the portion of the sidewall of the second support. The width or sidewall of the first support being larger than the width or sidewall of the second support provides a pocket or space that allows the supports to be slidably movable relative to each other. Shifting or sliding the supports into the space or pocket disengages the ratchet engagement of the tooth or teeth of the first and second supports allowing the jaws to open or spread apart. As such, the first support is configured with a sufficient width to accommodate the longitudinal travel of the second support and the lateral travel of the second support into the space within the first support. Also, the teeth on the first and second supports are configured with a sufficient width to engage when the first and second supports are aligned and to disengage or lose contact with the other when the first and second supports are shifted or offset relative to each other.

Accordingly, the engagement of the tooth or teeth of the first support 45 with the teeth of the second support 46 permits ratchet-like movement of the first support 45 towards the second support 46 and the second support towards the first support, but prevents backing up or reversing direction when the supports are longitudinally aligned. Therefore, this engagement provides a one-way ratchet that holds the first and second jaws in a locked or set condition as the two jaws are brought or squeezed together by a surgeon's hand or through a surgical instrument, e.g., a grasper, as the first support 45 is moved along to one of the steps or teeth of the second support 46. The set condition is subsequently unlocked as the first jaw or second jaw is manipulated or squeezed in a direction that disengages the undercut step and the mating face of the adjoining member. For example, the first and second jaws 41, 42 and/or connectors 43, 44 when actuated, e.g., shifted or squeezed, cause the teeth 47 of the second support 46 to lose engagement with the tooth or teeth of the first support 45.

The normal state or tendency of the clamped or compressed tissue or organ to expand causes or forces the first and second jaws 41, 42 apart or open. At least one of the first or second supports 45, 46 of one of the connectors 43, 44, in one aspect, is configured to exert an outward spring-pressure on the assembly of the two jaws. For example, a spring, in one aspect, is disposed within the hollow portion of the first support or otherwise coupled to the first or second support to enhance the tendency of the first and second jaws 41, 42 to open towards a spaced position. At least one of the first or second supports 45, 46 of one of the connectors 43, 44, in one aspect, is configured to exert an inward spring-pressure upon the two jaws. For example, a spring, in one aspect, is coupled between the first and second jaws 41, 42 to bias or enhance tendency of the first and second jaws 41, 42 to close towards a proximate position.

Extension actuators 48, 49 respectively extend from the first jaw 41 and second jaw 42. By manipulating, e.g., squeezing or sliding, at least one of the extension actuators, the ratchet engagement of the first and second jaws release thereby allowing the jaws to open. In one aspect, the extension actuators have grooves, apertures, slots, texture, projections or an enlarged surface area to enhance gripping or actuation of the extension actuators 48, 49.

In one aspect, the ratchet engagement of the first and second jaws 41, 42 is a one-way engagement that cannot be disengaged to re-open the jaws. Otherwise stated, shifting or sliding the jaws does not allow the jaws to re-open. In one aspect, excising or otherwise removing the tissue between the clip, forcibly separating the jaws, or cutting or otherwise deforming the clip will allow the jaws to re-open.

In various aspects, the first and second atraumatic jaws are sized and configured to exert sufficient clamping pressure so as to provide hemostasis in the event that the clamped portion of tissue is cut or excised. The clamping force is supplied by direct pressure being applied. The clamping force is maintained or assisted by, in one aspect, with the jaws being locked or in a set condition and in one aspect by a spring disposed or attached to the supports or jaws biasing the jaws together or close towards a proximate position. In various aspects, the traction of the clip upon the tissue is provided by a mesh, woven or textured material that forms the atraumatic or tissue contacting surfaces of the first and second jaws. As such, the tissue contacting surfaces is tractive in nature and thereby does not slip off of the tissue. The tissue contacting surfaces also accommodates anatomical irregularities in the clamped tissue.

The tissue contacting surface, in one aspect, includes or is formed of a first material generally compliant and arranged to conform to the shape of irregularities along the clamping footprint. In one aspect, the material is silicone foam, gel or another similar soft, low durometer or compliant material. A second material, in one aspect, encompasses, surrounds or coats the first material and is sized and configured to provide enhanced traction between the jaws and the occluded tissue. The second material may comprise of a woven or braided sleeve or covering of a more rigid or hard composition than the first material. The combination of the first and second materials cooperate to form a tractive relationship between the jaws and the tissue and also separates the occlusive clamping force from the force required to maintain the clamping in a particular position without slipping or creeping to ultimately provide atraumatic occlusion.

In various aspects, the first and second jaws are fitted with inserts with silicon foam covered with a loosely woven fabric. The inserts are snap-fitted or adhesively bonded to the jaws. The inserts may be made of or include an additional fitting of non-conductive material such as plastic that include portions extending along the insert and over the jaws where the jaws may come into contact with an electrosurgical device. Addition of inserts can make the metal jaws compatible with electrosurgical devices and similarly removal of the inserts or another compatible fitting can make the metal jaws compatible with mechanical cutters. In one aspect, the jaws include a raised portion or lip along the inner periphery to further assist in guiding the cutting device. Also, such a lip, in one aspect, provides protection of an insert or tissue contacting surface from the cutting device.

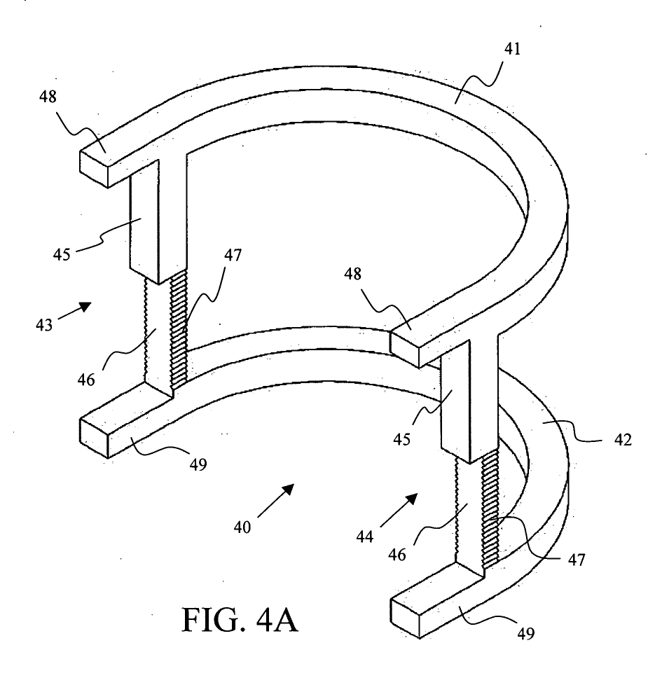

FIGS. 5A-5C illustrate a clip 50 similar to the clip shown in FIG. 1A-C. The clip 50 also has apertures, openings or slots 57, 58, in one of the jaws, e.g., second jaw 52. As the first jaw 51 and second jaw 52 are squeezed together towards a proximate position tissue there between is compressed. The teeth or steps of the connectors 53 and 54 provide a ratchet engagement between the jaws that holds the first and second jaws in a locked or set condition as the two jaws are brought or squeezed together by a surgeon's hand or through a surgical instrument, e.g., a grasper. As the first support 55 is moved to engage the steps or teeth of the second support 56, the first support 55 of each connector 53, 54 extends or traverses through the respective apertures 57, 58. To unlock the set condition or disengages the undercut step and the mating face of the first and second supports 55, 56, the extended first support 55 is manipulated or squeezed in a reverse or outward direction from the clamping or closing of the jaws. Therefore, the first supports 55 traverse back through the apertures 57, 58 to open the jaws and in one aspect incrementally opens as the jaws are in a set condition as the jaws are opened towards a spaced position through one or more intermediate set positions.

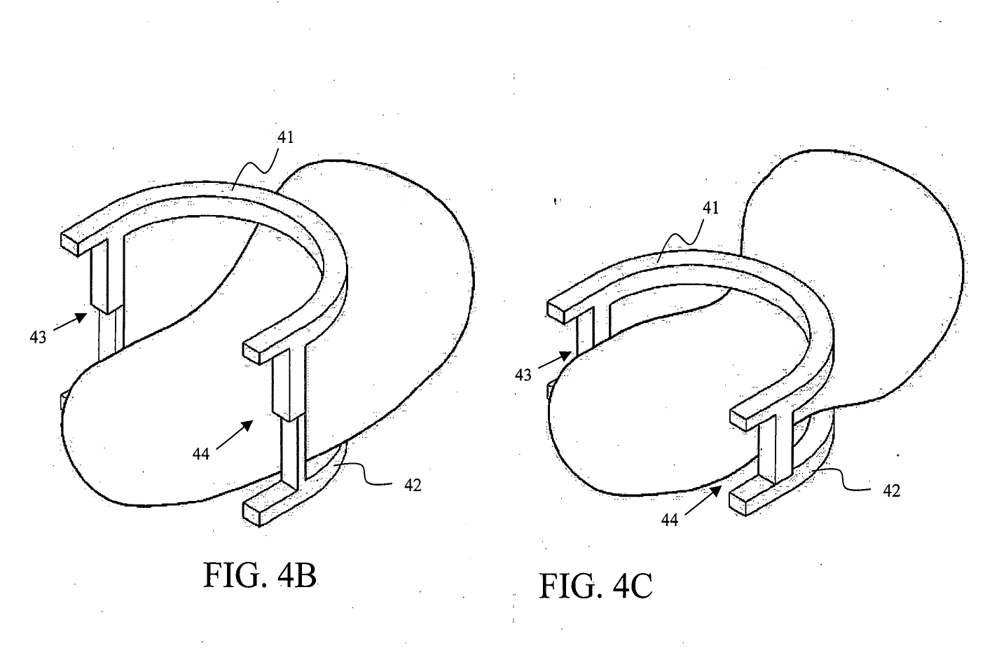

In reference to FIGS. 6A-E, the first and second jaws 61, 62 or portions thereof are of an electrically non-conductive construction. In one aspect, the jaws are made of plastic. As such, an electrosurgical tissue-cutting instrument 63 may be used in conjunction with the clip 60 to excise the portion of tissue within the curved jaw portions without interference, e.g., unintended or adverse reactions, from the first and second jaws. The combination of the clamp pressure and the electrosurgical hemostasis excise of significant portions of blood-rich organs without excessive bleeding may be accomplished.

In one aspect, jaws 61, 62 are sized and configured to provide a guide and/or insulator to be compatible with an electrosurgical cutting instrument. One such electrosurgical cutter 63 has a forked body member and an electrosurgically energized wire 64 extending between the ends of the legs of the forked body. The electrosurgical wire 64 may be placed against the surface of the clip and urged through the tissue as the wire is energized (FIGS. 6B-6E). The jaws 61, 62 are used as a guide and/or a heat sink for the heat generated by the electrosurgical discharge. Extended legs of the forked body member may extend slightly beyond the electrosurgical wire so as to provide additional guidance and control of the electrosurgical cutter. As such, the extended legs ride along the upper and lower surfaces of the clip as the wire rides along an inner face or surface of the first and second jaws.

In one aspect, the jaws include an electrically conductive portion in electrical continuity with a portion of the clamped tissue and also an insulated or non-conductive exposed face. At least one of the jaws is connected to the return path of the electrosurgical generator. An electrosurgical cutting instrument operating in a mono-polar mode may be energized adjacent to the electrically conductive jaw. The current density along the cutting instrument is much higher than the current density along the clamping surfaces. As such, a current path is established between the cutting instrument and the clip where the cutting element provides the electrosurgical effect and the clip acts as a return electrode. In this configuration, the current path is short and relatively unimpeded by a typical resistance of a long current path.

In one aspect a quasi bi-polar arrangement is achieved in a plastic electrically non-conductive clip when an electrically conducive portion is provided along the tissue engaging or contacting surfaces of the clip and a connection to the return path of the electrosurgical generator is provided. Additionally, a direct connection to an electrosurgical generator may be provided to the clip so that it may be energized to provide hemostasis. For example, an electrosurgical occlusion clip in one aspect includes a first jaw connected to a first electrical pole of a bipolar generator and a second jaw insulated from the first jaw is connected to a second pole of the electrosurgical generator. When energized, an electrosurgical current path is established between the jaws and through the tissue compressed between them. An alternate aspect of the electrosurgical clip comprises a first jaw and a second jaw in electrical continuity and connected to the mono-polar output of an electrosurgical generator. When the clip is energized, a current path is established between the occluded tissue and the electrosurgical grounding element. The high current density along with the clamping force between the jaws of the clip provides hemostasis within the occluded tissue.

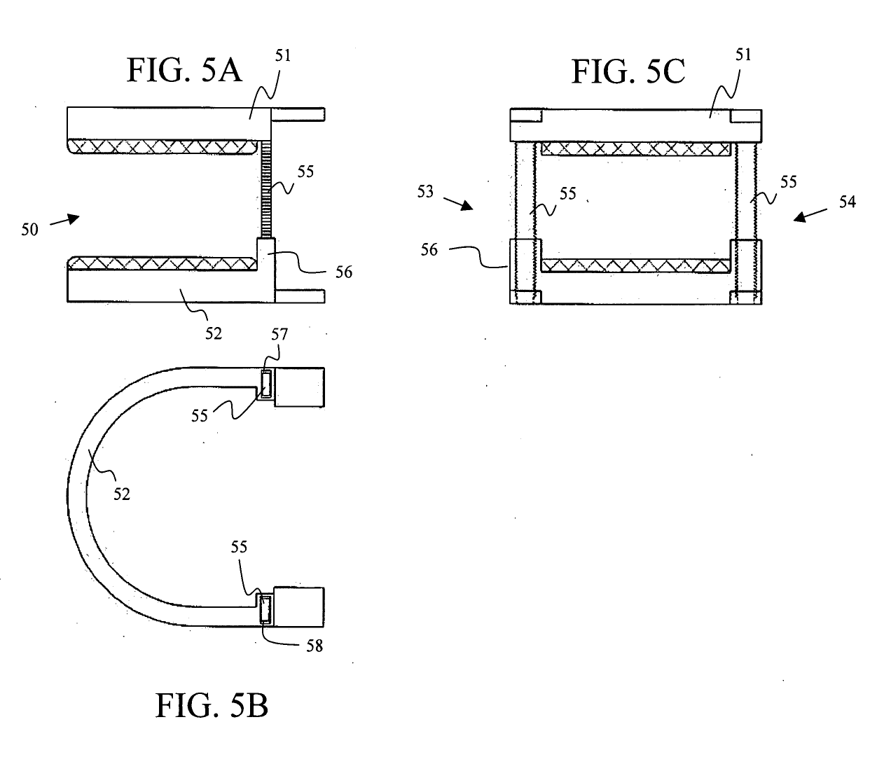

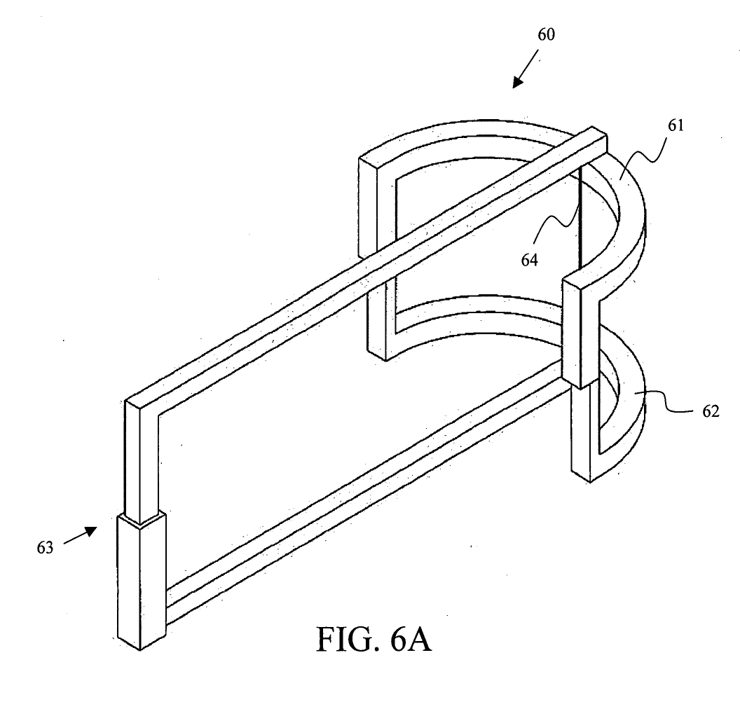

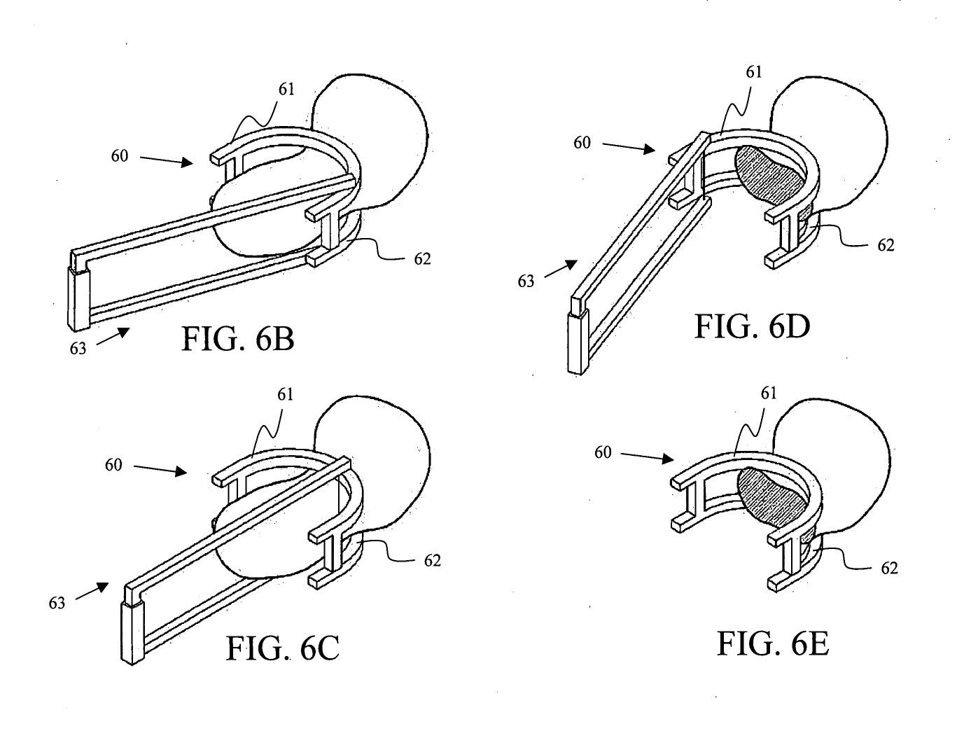

FIGS. 7-13 illustrate various other aspects clip or clips in accordance with the present invention and in which the first and second jaws 80, 90 may have various shapes and sizes. In one aspect, the shape, size and/or configuration of the jaws are for accommodating specific organs or for specific procedures. In one aspect, malleable or bendable jaws are provided for clamping specific portions or excluding specific portions of clamped tissue. Such jaws, in one aspect, include ball and sockets, shape memory material or flexible inner and rigid or stiff outer members to provide bendable and shapeable jaws. The shapeable or dynamically formable jaws may be introduced into the body cavity in a first shape or condition and subsequently and continuously reshaped into another shape as desired for the procedure or to isolate specific portions of an organ or tissue. In one instance, the first condition of the shapeable clip may be straight or substantially compact and linear to be introduced through an access port, e.g., a surgical trocar or cannula, or incision having a small width or diameter, e.g., 5 mm-12 mm. The clip subsequently may be formed or shaped as desired after being introduced through the access port.

In one aspect, the first and second jaws 80, 90 of the clip are metal. As such, a mechanical cutting instrument may be urged to make an incision along the path outlined by the jaws. In another aspect, the jaws are constructed of a material or include a material with weaken/flexible and strong/stiff portions that induce or allow bending or deforming of the jaws in a single plane. For example, the jaws may have a rectangular cross-section such that the preferred bending is in a plane parallel to the axis of the hinges of the jaws. As such, the strength of the jaws in the clamping or first direction is preserved while providing a shapeable or formable occlusion footprint.

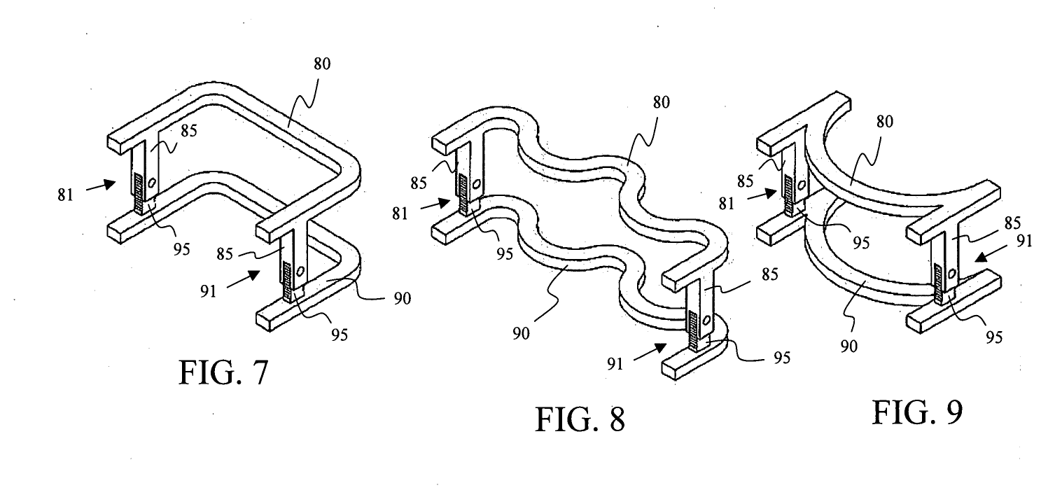

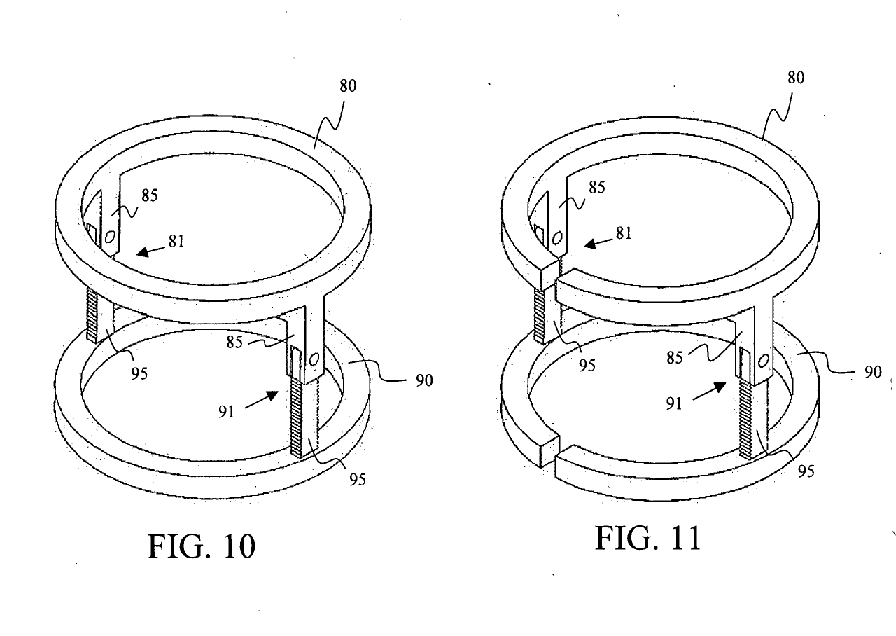

In one aspect, the clip is configured to isolate or clamp the tissue or portion of the tissue in order to spare as much healthy tissue as possible from the trauma of clamping. The shape of the clip in one aspect allows malignant tissue to be isolated prior to excision. As such, less seeding of cancer cells, for example, may result when the malignant masses are being treated. For example, the first and second jaws 80, 90 are generally curved, semi-circular or “C” shaped as shown in FIGS. 7-9. In another aspect, the first and second jaws 80, 90 are generally circular as shown for example in FIGS. 10-11. Supports 85 of the first jaw 80 is sized and configured to receive the supports 95 of the second jaw 90 and provide a pivoting and sliding relationship between the jaws. Hinge portions or points 81, 91 along with raised or recessed projections, steps or teeth on the supports 85, 95 are sized and configured to cooperate with a series of undercut steps along the opposing support such that as the jaws are squeezed or brought together from a spaced position to a proximate position through one or more intermediate set positions, the first jaw 80 is urged to step down and into the undercut faces of the steps of the second jaw 90 to form a locked relationship between the first jaw 80 and the second jaw 90. Pivoting the first and second jaws 80, 90 releases the locked or engaged relationship.

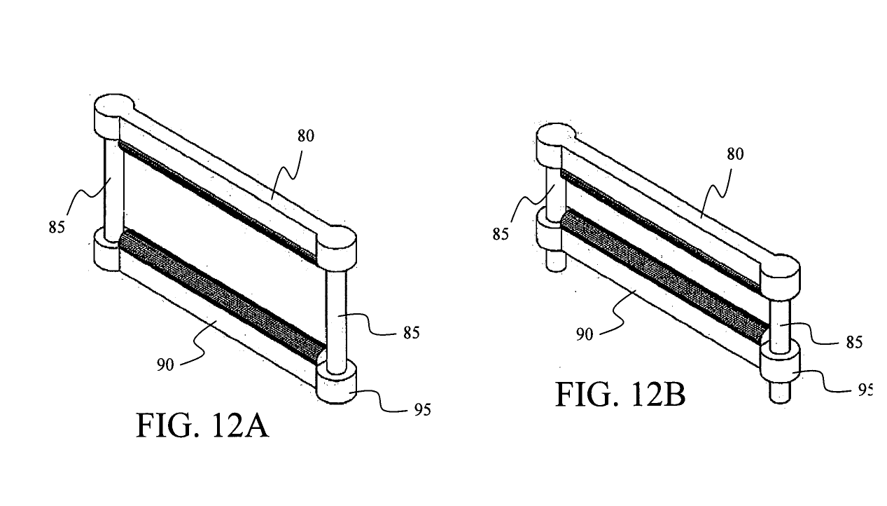

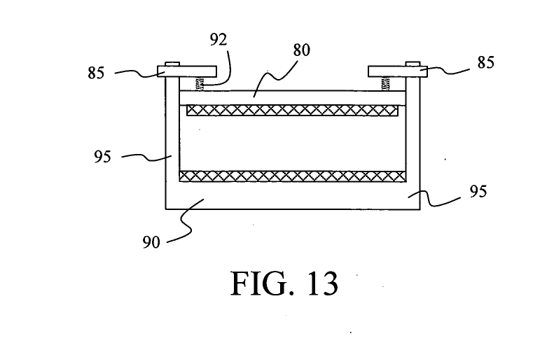

In one aspect, the first and second jaws 80, 90 are parallel and equidistant from each other thereby eliminating regions of excessive pressure that may cause tissue necrosis as shown for example in FIGS. 12-13. Friction between supports or raised/recessed steps, projections or teeth cooperating with corresponding undercut steps, projections or teeth provide incremental locking as the jaws are squeezed or brought together from a spaced position to a proximate position through one or more intermediate set positions. Pivoting or otherwise shifting or disengaging the ratchet engagement releases the locking or engaging relationship between the jaws. In one aspect, the jaws are unlocked or opened by driving the supports extending through apertures of the jaw in an opposite or second direction and in another aspect one or more bias or spring devices 92 are coupled to one of the jaws and/or support to induce the jaws apart or together.

Various aspects of the surgical clip may be used in an open surgical procedure, for example, cross-clamping a human kidney to exclude an area to be treated, excised or removed. The cross-clamp maintains isolation and hemostasis as the target tissue is removed and until another form of hemostasis is performed. The clip is introduced to the target tissue or organ through a large incision. The clip is place upon a human kidney as would be the case in a partial-nephrectomey surgical procedure. In this procedure a diseased portion of the kidney is removed and as much of the healthy kidney as possible is left. The tissue is very delicate and the collecting system within the kidney is spared insult by using atraumatic tissue contacting surfaces of the clip. A typical large cross-clamp, such as a vascular side-bite clamp or a bowl clamp can work if properly padded, but is large and obstructive.

The various aspects of clips previously described are also compatible with the cutting device used for excision. For example, the clip can be made of non-conductive material and not stainless steel as most surgical devices are in which an electrosurgical knife is often not compatible with such devices. The atraumatic nature of the clip is provided in one aspect by padding made of rubber or foam. The clip protects the padding by guiding the knife to prevent cutting into the padding and leaving pieces of the material behind. Additionally, the clip of various aspects being compact and shaped to guide the cutting device does not interfere with the view angle of a scalpel or other cutting devices.

For hand assisted laparoscopic procedures, one hand of a surgeon is inserted into a body cavity of a patient. The surgeon may carry devices and instruments into the body cavity through a “hand-port” that would otherwise not fit through a laparoscopic trocar, but an incision far smaller than one used in open procedures is used. Due to the size, configuration and simple operation of the various aspects of the clip described above, the clip may be carried into the body cavity and placed upon tissue to be occluded by the hand of the surgeon. In one aspect, the clip is carried into place by hand and subsequently connected to or grasped by a laparoscopic instrument of placement. For example, during a partial-nephrectomy, the clip may be folded to a small closed condition and carried by hand through the hand-port into the abdomen of a patient. The clip may be connected to a laparoscopic grasper so that the hand that carried it into the abdomen may release the clip. The liberated hand may grasp the target tissue, e.g., the lower lobe of a kidney, and guide the clip onto the tissue stereo tactically.

For a laparoscopic surgical procedure, the nature of laparoscopic instruments is linear so that the curved jaws and surfaces of open procedure instruments are not available since they do not fit through a trocar or a small incision or access port. As such, the choice of instrument is often limited to those that can be introduced through a rigid tubular port typically ranging in diameter from 5 mm to 12 mm for abdominal surgeries. However, closely mimicking the utility of conventional surgical instruments is often desired. Also, the various aspects of the clips described herein do not sacrifice the strength of other occlusion devices and/or any atraumatic characteristics to accommodate insertion and operation through small trocar ports. Various aspects of the previously described clips are sized and configured to be introduced through limited diameter ports, e.g., 5 mm to 12 mm. With releasable ratchet engagements, locking relationships between jaws provide sufficient and reliable strength to occlude or clamp tissue without interfering and assist in guiding other laparoscopic instrumentation utilized in the surgical procedure. For example, the clip delineates an outline or pattern to follow in excising of the tissue or organ.

Accordingly, the present invention provides an occlusion atraumatic guide clip and methods thereof. Although this invention has been described in certain specific embodiments, many additional modifications and variations would be apparent to those skilled in the art. It is therefore to be understood that this invention may be practiced otherwise than specifically described, including various changes in the size, shape and materials, without departing from the scope and spirit of the present invention. Also, as used herein, the term clip refers not only to surgical clips, but also to clamps, clinches and other similar types of surgical devices adapted for use in the previously described aspects and operations. Thus, embodiments of the present invention should be considered in all respects as illustrative and not restrictive, the scope of the present invention to be determined by the appended claims and their equivalents rather than the foregoing description.

1. A surgical guide clip comprising:
a first connector having a first support and a second support, the first support connected to the second support;
a second connector having a third support and a fourth support, the third support connected to the fourth support;
a first jaw having a tissue contacting surface, a first end and a second end, the first support extending from the first end of the first jaw, the third support extending from the second end of the first jaw, and the first and third supports extending generally perpendicularly to the tissue contacting surface of the first jaw; and
a second jaw having a tissue contacting surface, a first end and a second end, the second support extending from the first end of the second jaw, the fourth support extending from the second jaw, and the second and fourth supports extending generally perpendicularly to the tissue contacting surface of the second jaw.

2. The clip of claim 1 wherein the first support is slidably attached to the second support and the third support is slidably attached to the fourth support.

3. The clip of claim 1 wherein the first support is pivotably attached to the second support and the third support is pivotably attached to the fourth support.

4. The clip of claim 1 wherein one of the first support has a ratchet engagement with the second support and the third support has a ratchet engagement with the fourth support.

5. The clip of claim 4 wherein the ratchet engagement permits movement of the first jaw towards the second jaw to a set position and prevents movement of the first jaw away from the second jaw from the set position with the first support being axially aligned with the second support and the third support being axially aligned with the fourth support.

6. The clip of claim 5 wherein the ratchet engagement permits movement of the first jaw away from the second jaw with the first support being pivoted relative to the second support and the third support being pivoted relative to the fourth support.

7. The clip of claim 1 wherein the first support has an inner hollow portion and a projection extending from the inner hollow portion and the second support has teeth extending laterally from the second support to correspondingly engage with the projection of the first support.

8. The clip of claim 7 wherein the inner hollow portion is configured to substantially enclose the second support, the second support being slidable within the inner portion of the first support.

9. The clip of claim 6 wherein the ratchet engagement permits incremental movement in a first direction while the first support is axially aligned with the second support.

10. The clip of claim 9 wherein the ratchet engagement permits free movement in a second direction opposite the first direction when the second support is out of alignment with the second support.

11. The clip of claim 1 further comprising a pin extending through apertures in the first and second supports and a pin slot extending axially along the first and second supports.

12. A surgical guide clip comprising:
a first jaw having a first end, a second end and a tissue contacting surface extending from the first end to the second end;
a second jaw having a first end, a second end and a tissue contacting surface extending from the first end to the second end, the first jaw and the second jaw movable between a spaced position and a proximate position;
a first connector coupled to the first end of the first jaw and the first end of the second jaw and having a first projection operatively engageable with a second projection to regulate movement of the first jaw and second jaw between the spaced and proximate position; and
a second connector coupled to the second end of the first jaw and the second end of the second jaw.

13. The clip of claim 12 wherein the first connector further comprises a first support having a proximal end coupled to the first end of the first jaw and a distal end and a second support having a distal end coupled to the first end of the second jaw and a proximal end coupled to the distal end of the first support.

14. The clip of claim 13 wherein the second connector further comprises a third support coupled to the first jaw and a fourth support coupled to the second jaw, the third and fourth supports being generally parallel to the first and second supports and the first and third supports being movable a predetermined distance in a predetermined direction, as the first and second jaws are moved towards the proximate position.

15. The clip of claim 14 further comprising a first pin and a first pin slot and wherein the first support extends orthogonally from the first jaw and the distal end of the first support is connected to the second support extending orthogonally from the second jaw by the first pin slidably and pivotably connected to the first pin slot extending along the second support.

16. The clip of claim 15 wherein the first support has an interior portion and the second support slides within the interior portion as the first and second jaws are moved to the proximate position; and wherein the first projection extends laterally from a first wall of the interior portion and the second projection extends laterally from a first wall of the second support, the first projection extending towards the first wall of the second support and the second projection extending towards the first wall of the first support with the first and second jaws in a set position.

17. The clip of claim 16 wherein the first support has a second wall on the opposing side of the first support relative to the first wall and the second support has a second wall on the opposing side of the second support relative to the first wall, the second wall of the first support and the second wall of the second support operatively in contacting with each other when the first projection and the second projection operatively disengage from each other.

18. The clip of claim 12 wherein the first connector has a first hinge point pivotably coupling the first end of the second jaw to the first end of the first jaw and the second connector has a second hinge point pivotably coupling the second end of the second jaw to the second end of the first jaw.

19. The clip of claim 12 wherein the first and second connectors are symmetrical and have pivot pins disposed through pivot slots in each connector pivotably coupling the first jaw to the second jaw to allow opening of the jaws to the spaced position by allowing the first and second jaws to pivot in a direction opposite of the first direction.

20. The clip of claim 19 wherein the pivot slot has a width and length sized and configured to accommodate sufficient pivoting of the first and second jaws to disengage the first projection from the second projection.

21. The clip of claim 12 wherein the first jaw forms one of a circular, arcuate, and rectangular shapes and the second jaw is symmetrical to the first jaw.

22. The clip of claim 21 further comprising atraumatic surface covers removably connected on the tissue contacting surfaces of the first and second jaws.

23. The clip of claim 22 wherein the first jaw has a raised portion extending along the first jaw proximate the tissue contacting surface.

24. The clip of claim 12 wherein the first jaw is malleable and configurable to form multiple shapes.

25. The clip of claim 16 further comprising a spring disposed within the interior portion of the first support.

26. The clip of claim 16 further comprising an extension actuator extending from one of the first end of the first jaw, the second end of the first jaw, the first end of the second jaw and the second end of the second jaw.

27. The clip of claim 26 wherein the extension actuator has grooves to enhance actuation of the extension actuator and pivoting of the first and second jaws.

28. The clip of claim 12 wherein the first jaw and second jaw are sized and configured to be grasped by forceps.

29. A surgical guide clip comprising:
means for atraumatically securing tissue between a first tissue contacting surface and a second contacting surface and movable between a spaced position and a proximate position;
means for controlling movement of the means for atraumatically securing tissue between the spaced position and the proximate position through a plurality of intermediate set positions between the spaced position and the proximate position; and
means for releasing the means for controlling movement to permit movement of the means for atraumatically securing tissue from the proximate position to the spaced position bypassing at lease one of the plurality of intermediate set positions.

30. The clip of claim 29 wherein means for controlling movement comprising a ratchet engagement of a plurality of projections disposed to delimit each intermediate set position.

31. The clip of claim 29 means for releasing comprising a plurality of pivot pins connected to the means for atraumatically securing tissue and allowing disruption of alignment of the means for atraumatically securing tissue.
